(19) United States
(12) Patent Application Publication
(10) Pub. No.: US 2023/0057957 A1
(43) Pub. Date: Feb. 23, 2023

(54) UMBILICAL CORD MESENCHYMAL STEM CELLS FOR TREATMENT OF CHRONIC OBSTRUCTIVE PULMONARY DISEASE AND LUNG DEGENERATION

(71) Applicant: Therapeutic Solutions International, Inc., Oceanside, CA (US)

(72) Inventors: Thomas E. ICHIM, Oceanside, CA (US); Timothy G. DIXON, Oceandide, CA (US); James VELTMEYER, Oceanside, CA (US); Famela RAMOS, Oceanside, CA (US)

(73) Assignee: Therapeutic Solutions International, Inc., OCEANSIDE, CA (US)

(21) Appl. No.: 17/821,754

(22) Filed: Aug. 23, 2022

Related U.S. Application Data
(60) Provisional application No. 63/235,854, filed on Aug. 23, 2021.

(51) Int. Cl. A61K 35/51; A61K 38/20; C12N 5/0775

(52) U.S. Cl. CPC A61K 35/51; A61K 38/2013; C12N 5/0665

(57) ABSTRACT

The invention discloses means of treating lung degenerative diseases including chronic obstructive pulmonary disease (CODP) using umbilical cord mesenchymal stem cells such as JadiCells alone, and/or using said cells under conditions that are activated in order to endow enhanced regenerative activity. In one embodiment said activation of said mesenchymal stem cells is performed through stimulation with a toll like receptor agonist at a concentration and duration sufficient to induce a >50% increase in keratinocyte growth factor expression from said stem cells. In another embodiment the invention provides the use of JadiCells as a means of producing exosomes, wherein said exosomes possess therapeutic properties capable of reducing inflammation, fibrosis and degeneration associated with COPD, as well as stimulation of regenerative activity. In some JadiCells are activated by a treatment with Activated Protein C.

### CROSS-REFERENCE TO RELATED APPLICATIONS

This application claims the benefit of priority to United States Provisional Application Serial No. 63/235,854, entitled “Umbilical Cord Mesenchymal Stem Cells for Treatment of Chronic Obstructive Pulmonary Disease and Lung Degeneration”, filed on Aug. 23, 2021, which is incorporated by reference herein in its entirety

### FIELD OF THE INVENTION

The invention pertains to the area of treating chronic obstructive pulmonary disease and lung degeneration, more particularly the invention pertains to the use of mesenchymal stem cells that have been enhanced for said treatment.

### BACKGROUND

Chronic Obstructive Pulmonary Disease (COPD), an umbrella term covering chronic bronchitis and emphysema, is the fourth largest cause of death in the United States and is projected to be the third by 2020 [1]. COPD is associated with an exaggerated chronic inflammatory response which is responsible for the airway abnormalities such as constriction and architectural distortion of the lung parenchyma. Patients generally undergo a progression of declining lung function, characterized by intensification of cough, shortness of breath, and sputum production. Extrapulmonary manifestations of COPD include osteoporosis, cardiovascular disease, skeletal muscle abnormalities, and depression [2]. Chronic obstructive pulmonary disease (COPD) is a significant cause of morbidity and mortality worldwide. In contrast to other chronic diseases, COPD is increasing in prevalence.. The costs to society for treating COPD are high, accounting for approximately 3.4% of the total health care budget of the European Union. In the United States, the direct and indirect costs of COPD are estimated to be more than $30 billion.

It is known that 30% of patients with COPD have elevated levels of eosinophils in the airway as measured by sputum induction or bronchoalveolar lavage. In COPD, the response to oral and inhaled corticosteroids (ICS) is related to the intensity of the airway eosinophilic inflammation, and a sputum eosinophilia count of greater than 3% has been demonstrated to be a good predictor of response to steroids in COPD. A strategy in which increasing therapy with corticosteroids was used to control sputum eosinophilia greater than 3% in COPD resulted in a reduction in the frequency of severe COPD exacerbations requiring admission to a hospital when patients were stepped up to oral corticosteroid therapy. Standard therapy for acute exacerbations of COPD (AECOPD) includes treatment of inflammation with systemic corticosteroids, which are associated with a reduction in length of hospital stay and hastened recovery. Corticosteroids are responsible for early apoptosis of eosinophils and generally result in a reduction in eosinophilia. Unfortunately, long-term therapy with corticosteroids is associated with significant side effects such as suppression of the hypothalamic-pituitary-adrenal axis and osteoporosis, and corticosteroids do not avert exacerbations in all eosinophilic COPD patients. COPD patients with increased sputum eosinophil counts have been shown to have significant improvements in forced expiratory volume in 1 second (FEV.sub.1) and quality of life-scores that were associated with decreased sputum eosinophil counts and eosinophil cationic protein (ECP) levels. Thus, therapies specifically targeted at eosinophils in COPD may have beneficial effects.

Current treatments for COPD are primarily palliative and are based on severity of disease. According to the Global Strategy for the Diagnosis, Management, and Prevention of COPD (GOLD) guidelines, the following treatments are recommended: Stage I, which is characterized by mild obstruction, the aim is to reduce risk factors associated with exacerbations, for example by providing flu vaccine and use of short-acting bronchodilator as needed. Stage II patients are classified as moderate obstruction, where risk factors are to be reduced by vaccination, and the use of long-acting bronchodilators, as well as cardiopulmonary rehabilitation is advised in addition to short-acting bronchodilators. Patients with Stage III disease are considered to suffer from severe obstruction, in which inhaled glucocorticoids are added to the regime of Stage II. In Stage IV, which is considered very severe obstruction or moderate obstruction with evidence of chronic respiratory failure, long-term oxygen therapy is added, as well as consideration of surgical options such as lung volume reduction surgery and lung transplantation [3].

### SUMMARY

Preferred embodiments are directed to methods of treating a lung degenerative disease comprising administration of a therapeutic cell, wherein said therapeutic cell is generated by the steps of: a) obtaining umbilical cord mesenchymal stem cells; b) culturing said mesenchymal stem cells in a liquid media capable of allowing for proliferation of said mesenchymal stem cells; c) extracting from said single cell suspension cells expressing the markers CD31 and CD73; and d) priming said cells with an agent capable of augmenting production of lung regenerative properties of said cells.

Preferred embodiments include methods wherein said lung regenerative properties are selected from a group comprising of: a) inhibiting inflammation; b) enhancing renewal of pulmonary progenitor cells; c) inhibiting pulmonary fibrosis; and d) preventing apoptosis of pulmonary cells.

Preferred embodiments include methods wherein said inflammatory cytokines are associated with increasing permeability of blood vessels.

Preferred embodiments include methods wherein said inflammatory cytokines are associated with induction of hypotension.

Preferred embodiments include methods wherein said inflammatory cytokines are associated with induction of vascular leakage.

Preferred embodiments include methods wherein said inflammatory cytokines are associated with an increase in pro-thombotic molecules on the vasculature.

Preferred embodiments include methods wherein said lung regenerative cytokine is keratinocyte growth factor.

Preferred embodiments include methods wherein said lung regenerative cytokine is ciliary neurotrophic growth factor.

Preferred embodiments include methods wherein said inhibition of fibrosis is achieved by augmentation of matrix metalloprotease activity.

Preferred embodiments include methods wherein said inhibition of apoptosis is accomplished by production of IGF-1.

Preferred embodiments include methods wherein said inhibition of apoptosis is accomplished by production of VEGF.

Preferred embodiments include methods wherein said mesenchymal stem cell are activated by exposure to a toll like receptor agonist

Preferred embodiments include methods wherein said toll like receptor is TLR-1.

Preferred embodiments include methods wherein said activator of TLR-1 is Pam3CSK4.

Preferred embodiments include methods wherein said toll like receptor is TLR-2.

Preferred embodiments include methods wherein said activator of TLR-2 is HKLM.

Preferred embodiments include methods wherein said toll like receptor is TLR-3.

Preferred embodiments include methods wherein said activator of TLR-3 is Poly:IC.

Preferred embodiments include methods wherein said toll like receptor is TLR-4.

Preferred embodiments include methods wherein said activator of TLR-4 is LPS.

Preferred embodiments include methods wherein said activator of TLR-4 is Buprenorphine.

Preferred embodiments include methods wherein said activator of TLR-4 is Carbamazepine.

Preferred embodiments include methods wherein said activator of TLR-4 is Fentanyl.

Preferred embodiments include methods wherein said activator of TLR-4 is Levorphanol.

Preferred embodiments include methods wherein said activator of TLR-4 is Methadone.

Preferred embodiments include methods wherein said activator of TLR-4 is Cocaine.

Preferred embodiments include methods wherein said activator of TLR-4 is Morphine.

Preferred embodiments include methods wherein said activator of TLR-4 is Oxcarbazepine.

Preferred embodiments include methods wherein said activator of TLR-4 is Oxycodone.

Preferred embodiments include methods wherein said activator of TLR-4 is Pethidine.

Preferred embodiments include methods wherein said activator of TLR-4 is Glucuronoxylomannan from Cryptococcus.

Preferred embodiments include methods wherein said activator of TLR-4 is Morphine-3 -glucuronide.

Preferred embodiments include methods wherein said activator of TLR-4 is lipoteichoic acid.

Preferred embodiments include methods wherein said activator of TLR-4 is β-defensin 2.

Preferred embodiments include methods wherein said activator of TLR-4 is small molecular weight hyaluronic acid.

Preferred embodiments include methods wherein said activator of TLR-4 is fibronectin EDA.

Preferred embodiments include methods wherein said activator of TLR-4 is snapin.

Preferred embodiments include methods wherein said activator of TLR-4 is tenascin C.

Preferred embodiments include methods wherein said toll like receptor is TLR-5.

Preferred embodiments include methods wherein said activator of TLR-5 is flagellin.

Preferred embodiments include methods wherein said toll like receptor is TLR-6.

Preferred embodiments include methods wherein said activator of TLR-6 is FSL-1.

Preferred embodiments include methods wherein said toll like receptor is TLR-7.

Preferred embodiments include methods wherein said activator of TLR-7 is imiquimod.

Preferred embodiments include methods wherein said toll like receptor of TLR-8.

Preferred embodiments include methods wherein said activator of TLR8 is ssRNA40/LyoVec.

Preferred embodiments include methods wherein said toll like receptor of TLR-9.

Preferred embodiments include methods wherein said activator of TLR-9 is a CpG oligonucleotide.

Preferred embodiments include methods wherein said activator of TLR-9 is ODN2006.

Preferred embodiments include methods wherein said activator of TLR-9 is Agatolimod.

Preferred embodiments include methods wherein said umbilical cord mesenchymal stem cell is an isolated cell prepared by a process comprising: placing a subepithelial layer of a mammalian umbilical cord tissue in direct contact with a growth substrate; and culturing the subepithelial layer such that the isolated cell from the subepithelial layer is capable of self-renewal and culture expansion, wherein the isolated cell expresses at least three cell markers selected from the group consisting of CD29, CD73, CD90, CD166, SSEA4, CD9, CD44, CD146, or CD105, and wherein the isolated cell does not express NANOG and at least five cell markers selected from the group consisting of CD45, CD34, CD14, CD79, CD106, CD86, CD80, CD19, CD117, Stro-1, or HLA-DR.

Preferred embodiments include methods wherein the isolated cell expresses CD29, CD73, CD90, CD166, SSEA4, CD9, CD44, CD146, and CD105.

Preferred embodiments include methods wherein the isolated cell does not express CD45, CD34, CD14, CD79, CD106, CD86, CD80, CD19, CD117, Stro-1, and HLA-DR.

Preferred embodiments include methods wherein the isolated cell is positive for SOX2.

Preferred embodiments include methods wherein the isolated cell is positive for OCT4.

Preferred embodiments include methods wherein the isolated cell is positive for SOX2 and OCT4.

Preferred embodiments include methods wherein the wherein the isolated cell is capable of differentiation into a cell type selected from the group consisting of adipocytes, chondrocytes, osteocytes, cardiomyocytes, endothelial cells, and myocytes.

Preferred embodiments include methods wherein the isolated cell produces exosomes expressing CD63, CD9, or CD63 and CD9.

Preferred embodiments include methods wherein culturing comprises culturing in a culture media that is free of animal components.

Preferred embodiments include a culture of differentiated cells derived from an isolated cell, wherein the culture of differentiated cells includes a cell type selected from the group consisting of adipocytes, chondrocytes, osteocytes, cardiomyocytes, endothelial cells, myocytes and combinations thereof.

Preferred embodiments include methods wherein the isolated cell has been differentiated into an adipocyte cell.

Preferred embodiments include methods wherein the isolated cell has been differentiated into a chondrocyte cell.

Preferred embodiments include methods wherein the isolated cell has been differentiated into an osteocyte cell.

Preferred embodiments include methods wherein the isolated cell has been differentiated into a cardiomyocyte cell.

Preferred embodiments include methods wherein the isolated cell has been expanded into a cell culture.

Preferred embodiments include methods wherein IL-2 is administered together with said mesenchymal stem cells.

Preferred embodiments include methods wherein said IL-2 is administered at a concentration sufficient to induce generation of T regulatory cells.

Preferred embodiments include methods wherein said T regulatory cells express FoxP3.

Preferred embodiments include methods wherein said T regulatory cells express PD-1L

Preferred embodiments include methods wherein said T regulatory cells suppress death of type II pulmonary epithelial cells.

### BRIEF DESCRIPTION OF THE DRAWINGS

FIG. 1 is a bar graph comparing the effects of: a) adipose derived MSCs, b) bone marrow derived MSCS, and c )JadiCells on neutrophil levels in BALB/c mice that were administered elastase.

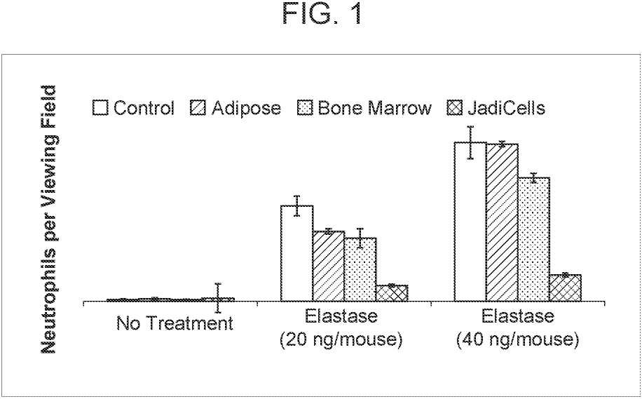

### DETAILED DESCRIPTION OF THE INVENTION

The invention teaches administration of umbilical cordl mesenchymal stem cells for treatment of COPD. In some cases administration of said mesenchymal stem cells is performed using cells that have been activated prior to administration. The process of activating said placentally derived mesenchymal is performed in order to augment regenerative, and/or anti-inflammatory, and/or migratory, and/or anti-apoptotic, and/or anti-fibrotic activities of said mesenchymal stem cells.

The invention teaches use of mesenchymal stem cell, in one particular embodiment, mesenchymal stem cell possessing initially CD34 and CD73, for treatment of COPD. In one particular embodiment, COPD is caused in party by cytokine upregulation, as well as subacute production of disseminated intravascular coagulation response causing degeneration of the alveoli. In one specific embodiment, mesenchymal stem cell are primed with an inflammatory or proinflammatory signal, in order to elicit a corresponding anti-inflammatory and pro-regenerative profile.

Within the context of the invention is the novel finding that prestimulation of mesenchymal stem cell with activated protein C (APC) is disclosed as a means of increasing anti-inflammatory potency of mesenchymal stem cell. Said anti-inflammatory potency may be utilized as a means of protecting animals or patients from COPD and inducing regeneration of pulmonary tissue.

The invention teaches means of selecting mesenchymalstem cell for enhanced efficacy based on expression of CD73, or lack of expression of certain proteins. Various terms are used to describe cells in culture. Cell culture refers generally to cells taken from a living organism and grown under controlled condition (“in culture” or “cultured”). A primary cell culture is a culture of cells, tissues, or organs taken directly from an organism(s) before the first subculture. Cells are expanded in culture when they are placed in a growth medium under conditions that facilitate cell growth and/or division, resulting in a larger population of the cells. When cells are expanded in culture, the rate of cell proliferation is sometimes measured by the amount of time needed for the cells to double in number. This is referred to as doubling time.

A cell line is a population of cells formed by one or more subcultivations of a primary cell culture. Each round of subculturing is referred to as a passage. When cells are subcultured, they are referred to as having been passaged. A specific population of cells, or a cell line, is sometimes referred to or characterized by the number of times it has been passaged. For example, a cultured cell population that has been passaged ten times may be referred to as a P10 culture. The primary culture, i.e., the first culture following the isolation of cells from tissue, is designated P0. Following the first subculture, the cells are described as a secondary culture (P1 or passage 1). After the second subculture, the cells become a tertiary culture (P2 or passage 2), and so on. It will be understood by those of skill in the art that there may be many population doublings during the period of passaging; therefore the number of population doublings of a culture is greater than the passage number. The expansion of cells (i.e., the number of population doublings) during the period between passaging depends on many factors, including but not limited to the seeding density, substrate, medium, growth conditions, and time between passaging.

A conditioned medium is a medium in which a specific cell or population of cells has been cultured, and then removed. When cells are cultured in a medium, they may secrete cellular factors that can provide trophic support to other cells. Such trophic factors include, but are not limited to hormones, cytokines, extracellular matrix (ECM), proteins, vesicles, antibodies, and granules. The medium containing the cellular factors is the conditioned medium.

Generally, a trophic factor is defined as a substance that promotes or at least supports, survival, growth, proliferation and/or maturation of a cell, or stimulates increased activity of a cell.

When referring to cultured vertebrate cells, the term senescence (also replicative senescence or cellular senescence) refers to a property attributable to finite cell cultures; namely, their inability to grow beyond a finite number of population doublings (sometimes referred to as Hayflick’s limit). Although cellular senescence was first described using mesenchymal stem cell-like cells, most normal human cell types that can be grown successfully in culture undergo cellular senescence. The in vitro lifespan of different cell types varies, but the maximum lifespan is typically fewer than 100 population doublings (this is the number of doublings for all the cells in the culture to become senescent and thus render the culture unable to divide). Senescence does not depend on chronological time, but rather is measured by the number of cell divisions, or population doublings, the culture has undergone. Thus, cells made quiescent by removing essential growth factors are able to resume growth and division when the growth factors are re-introduced, and thereafter carry out the same number of doublings as equivalent cells grown, continuously. Similarly, when cells are frozen in liquid nitrogen after various numbers of population doublings and then thawed and cultured, they undergo substantially the same number of doublings as cells maintained unfrozen in culture. Senescent cells are not dead or dying cells; they are actually resistant to programmed cell death (apoptosis), and have been maintained in their nondividing state for as long as three years. These cells are very much alive and metabolically active, but they do not divide. The nondividing state of senescent cells has not yet been found to be reversible by any biological, chemical, or viral agent.

As used herein, the term Growth Medium generally refers to a medium sufficient for the culturing of umbilicus-derived cells. In particular, one presently preferred medium for the culturing of the cells of the invention herein comprises Dulbecco’s Modified Essential Media (also abbreviated DMEM herein). Particularly preferred is DMEM-low glucose (also DMEM-LG herein) (Invitrogen, Carlsbad, Calif.). The DMEM-low glucose is preferably supplemented with 15% (v/v) fetal bovine serum (e.g. defined fetal bovine serum, Hyclone, Logan Utah), antibiotic s/antimycotics (preferably penicillin (100 Units/milliliter), streptomycin (100 milligrams/milliliter), and amphotericin B (0.25 micrograms/milliliter), (Invitrogen, Carlsbad, Calif.)), and 0.001% (v/v) 2-mercaptoethanol (Sigma, St. Louis Mo.). In some cases different growth media are used, or different supplementations are provided, and these are normally indicated in the text as supplementations to Growth Medium.

Also relating to the present invention, the term standard growth conditions, as used herein refers to culturing of cells at 37.degree. C., in a standard atmosphere comprising 5% CO.sub.2. Relative humidity is maintained at about 100%. While foregoing the conditions are useful for culturing, it is to be understood that such conditions are capable of being varied by the skilled artisan who will appreciate the options available in the art for culturing cells, for example, varying the temperature, CO.sub.2, relative humidity, oxygen, growth medium, and the like.

The use of mesenchymal stem cell for treatment of COPD may be based, in one embodiment of the invention, on the reduction of pathological immunological parameters associated with COPD. One of skill in the art is referred to publications supporting immunological mediated pathology in COPD [4], including specific demonstration that the destruction of alveolar tissue is associated with T cell reactivity [5, 6], pathological pulmonary macrophage activation [7], and auto-antibody production [8].

In one embodiment mesenchymal stem cell are utilized to treat COPD. Such cells are superior to conventional MESENCHYMALSTEM CELL, however, one of skill in the art is referred to publications regarding conventional MESENCHYMALSTEM CELL in order to direct on how to utilize placentally derived MESENCHYMALSTEM CELL in immune regulation including suppressing autoreactive T cells [9, 10], inhibiting macrophage activation [11], and also downregulating autoantibody responses [12]. The utilization of mesenchymalstem cell in regenerating lung tissue has been shown using other MESENCHYMALSTEM CELL in neonatal oxygen-damaged lungs, which results in COPD-like alveoli dysplasia, has been demonstrated to yield improvements in two recent publications [13, 14].

In some embodiments the treatment of COPD is performed prophylactically by administration activated mesenchymal stem cell. Initiation of COPD is believed to occur in many cases as result of noxious agents, particularly, but not exclusively in cigarette smoke. One established mechanism of initial alveolar injury involves smoke induced activation of inducible nitric oxide synthase (iNOS), which in turn produces cytotoxic free radicals such as peroxynitrite (ONOO⁻), which cause in mice a condition resembling emphesyma. Interestingly, mice lacking iNOS, or treated with a chemical inhibitor had some degree of protection from cigarette smoke induced pathology [15]. Thus in one embodiment, mesenchymal stem cell and/or derivatives thereof are administered in order to reduce peroxynitrite induced damage.

In other embodiments, said mesenchymal stem cell inhibit neutrophil activation. Studies using human neutrophils have shown that nicotine itself stimulates neutrophils to produce the inflammatory cytokine interleukin-8, in an iNOS-dependent manner [16]. Accordingly, in one embodiment said invention teaches the use of placental products for reduction of neurophil activation. Indeed the same study demonstrated that smokers possessed higher systemic levels of interleukin-8 as compared to non-smokers. This is correlated in patients with COPD which have higher inflammatory markers compared to controls, including TNF-alpha and IL-8 [17]. Another mechanism associated with COPD initiation is the generation of collagen degradation products such as the tripeptide chemoattractant N-acetyl Pro—Gly—Pro (PGP), which potently elicits neutrophil retention and activation. PGP is found in significantly higher concentrations in lavage samples of COPD patients as compared to controls, and also has been demonstrated to induce a COPD-like condition when administered into experimental animals [18]. Matrix metalloprotease (MMP)-9 has been demonstrated to be involved in the generation of PGP from collagen, and treatment of neutrophils with this agent stimulates their activation of MMP-9, thus suggesting an autostimulatory loop [19].

Inflammatory conditions stimulated by free radical stress and extracellular matrix degradation products stimulate various receptors within the lung to cause damage, and/or inhibit regeneration. For example, RTP801, is a protein that is inducible by HIF-1alpha, which causes death of alveolar cells in smoke induced lung injury models [20]. Yoshida et al demonstrated that Rtp801 transcript and protein are overexpressed in human emphysematous lungs and in lungs of mice exposed to cigarette smoke. Mechanistically, they found that Rtp801 was necessary and sufficient for NF-kB activation in cultured pulmonary cells and, when artificially expressed in mouse lungs by gene transfection, the protein promoted NF-kB activation, alveolar inflammation, oxidative stress and apoptosis of alveolar septal cells. Experiments furthermore demonstrated that mice lacking Rtp801 by means of gene knock-out were protected against acute cigarette smoke-induced lung injury. Protection was associated with increased mTOR signaling. Furthermore, the authors found that Rtp801 knockout mice were protected against emphysema when exposed chronically to cigarette smoke [21]. The mechanism of pulmonary damage associated with Rtp801 involves not only NF-kB associated induction of inflammatory cytokines but also ceramide-dependent apoptotic pathways. Specifically, it was demonstrated that direct lung instillation of either RTP801 expression plasmid or ceramides in mice triggered alveolar cell apoptosis and oxidative stress. RTP801 overexpression up-regulated lung ceramide levels 2.6-fold as compared to administration of a control plasmid. In turn, instillation of lung ceramides doubled the lung content of RTP801. Cell sorting after lung tissue dissociation into single-cell suspension showed that ceramide triggers both endothelial and epithelial cell apoptosis in vivo. It may be possible that endothelial apoptosis triggers a cascade of enhanced hypoxia, which in turn further augments HIF-1 alpha activation, thus self-perpetuating expression of RTP801. Interestingly, mice lacking rtp801 were protected against ceramide-induced apoptosis of epithelial type II cells, but not type I or endothelial cells [22]. This is of interest for two reasons, firstly, epithelial type II cells are known to be capable of acting as “regenerative cells” in the lung, which start proliferating after various injury signals [23], and secondly, the ceramide apoptotic pathway is triggered by various inflammatory signals associated with COPD such as TNF-alpha produced by neutrophils and monocytes [24-26].

In some embodiments, the mesenchymal stem cell are used to suppress “danger” signaling. Globally, RTP801 may be seen as a damage “sensor” molecule, amongst which other molecules such as toll like receptors (TLRs), and other activators of innate immune system play similar roles [27]. For example, TLR2, TLR3, and TLR4 have been found to be expressed in airway smooth muscle cells, which were activated by ligands found in inflammatory conditions associated with COPD and pulmonary remodeling such as extracellular matrix degradation products [28]. A clinical study performed mini-bronchoalveolar lavage (mini-BAL) on ten nonsmoker subjects without COPD, six smokers without COPD, and fifteen smokers with COPD. COPD mini-BAL showed increased neutrophil numbers, reduced neutrophil apoptosis, which was associated with increased TLR4 expression, compared with those in nonsmoker subjects without COPD. Demonstrating the importance of TLR4 was that in vitro administration of blocking antibodies to TLR4 resulted in increased neutrophil apoptosis [29]. Specific genetic variants of TLR4 have been associated with development of COPD, thus suggesting this molecule to also be a link to initiation and progression of the inflammatory state of this condition [30].

In addition to neutrophils, other cells of the innate immune system are associated with COPD. For example, natural killer (NK) cells have between found to be associated with initiation and progression of the disease. Wang et al performed a 124 patient study in smokers with COPD. They found systemic NK cell activation correlated with number of cigarettes smoked. Additionally, in induced sputum, the proportion of activated killer cells was related to disease state rather than current smoking status, with current and ex-smokers with COPD having significantly higher rates of activation than healthy smokers and healthy non-smokers [31]. NK activation is associated with production of cytotoxic factors such as granzyme, as well as various inflammatory cytokines including interferon gamma, which sensitize cells to inflammatory and immunologically mediated damage [32]. NK cell activation appears to be associated with recognition by the NK activating receptor NKG2D of the ligand RAET1ε, which is expressed on injured and stressed tissues. In fact, mice lacking NKG2D have been demonstrated to have a resistance to development of COPD-like pathology after exposure to cigarette smoke or viral infection [33]. While the natural function of NK cells in the lungs appears to be control of various infections [34], in the case of COPD it appears that these cells are “misguided” towards augmentation and self-perpetuation of the ongoing inflammatory cascade [35]. Thus in some embodiments of the invention, mesenchymal stem cell and derivatives thereof are utilized to suppress NK activity in COPD.

In other embodiments of the invention, T cells are modulated by mesenchymal stem cell /or derivatives thereof in order to inhibit, ameliorate, and in some cases reverse COPD. Contributions of adaptive immune cells to ongoing inflammatory processes has becoming increasingly recognized in situations such as ischemia/reperfusion injury [36], liver injury [37], and cancer [38], the situation of COPD is no exception. Suggesting a role for T cells in COPD was an early study in 1987 in which T lymphocytes were found to be significantly present in lavage fluid of patients with COPD but not controls. Furthermore, numbers of T cells were significantly reduced in responders to thiol drug tiopronin [39]. Indeed, other studies have confirmed the presence of various types of T cells, both CD4 and CD8 are present in abnormally high levels in COPD patients as compared to controls, with smoking augmenting levels of these cells [40-43]. Suggestive of a possible autoimmune activity of intrapulmonary T cells came from studies showing activated state of T cells in lungs of COPD patients. A study by Glader et al. examine peripheral blood lymphocytes from six never-smokers, eight smokers and 17 smokers with COPD. The number of lymphocytes per millilitre was higher in smokers than in never-smokers. No differences were found between the three groups in regard to proportions of lymphocyte populations, but the number of CD4+ T-cells in smokers was higher than in both never-smokers and COPD patients. The degree of T-cell activation was similar in all patient groups; however, a clear correlation between CD69 expression on CD4+ T-cells and lung function (FEV(1)% of predicted) was found when examining current smokers, with or without COPD [44]. Another study examined the Th1 associated transcription factor, STAT4, expression in lungs of patients with COPD. Th1 cells are associated with interferon gamma production and stimulation of inflammatory cascades, in part through macrophage activation and specifically stimulation of iNOS, as well as augmentation of NK activity. The study examined expression of STAT4, phospho-STAT4, IFN-gamma and T-box expressed in T-cells (T-bet) proteins in bronchial biopsies and bronchoalveolar lavage (BAL)-derived lymphocytes, obtained from 12 smokers with mild/moderate chronic obstructive pulmonary disease (COPD) (forced expiratory volume in one second (FEV1) 59 +/- 16% predicted), 14 smokers with normal lung function (FEV1 106 +/- 12% pred) and 12 nonsmoking subjects (FEV1 111 +/- 14% pred). In bronchial biopsies of COPD patients, the number of submucosal phospho-STAT4+ cells was increased (240 (22-406) versus 125 (0-492) versus 29 (0-511) cells mm(-2)) when compared with both healthy smokers and control nonsmokers, respectively. In smokers, phospho-STAT4+ cells correlated with the degree of airflow obstruction and the number of IFN-gamma+ cells. Similar results were seen in BAL (2.8 (0.2-5.9) versus 1.03 (0.09-1.6) versus 0.69 (0-2.3) lymphocytes x mL(-1) x 10(3)). In all smokers who underwent lavage, phospho-STAT4+ lymphocytes correlated with airflow obstruction and the number of IFNgamma+ lymphocytes [45].

In addition to CD4 activation, activation of CD8 T cells has been reported in COPD. An investigation of bronchoscopy with airway lavages and endobronchial mucosal biopsy sampling was performed in 35 patients with COPD, 21 healthy never-smokers and 16 smokers with normal lung function. Epithelial CD8+ lymphocyte numbers were higher in the COPD group compared to never-smoking controls. Among gated CD3+cells in BAL, the percentage of CD8+ NKG2D+ cells was enhanced in patients with COPD and smokers with normal lung function, compared to never-smokers. NKG2D is a receptor associated with stimulation of cytotoxic function by both NK cells and CD8 T cells. The percentage of CD8+ CD69+ cells and cell surface expression of CD69 were enhanced in patients with COPD and smokers with normal lung function, compared to never-smokers [46]. Given that both NKG2D and CD69 are associated with activation of CD8 cells, it is reasonable to believe that COPD is associated with an abnormality in the activation status of these cells.

In addition to activation of CD4 and CD8 T cells, there appears to be a deficiency in the T cells that are required to suppress rampant T cell activation, the T regulatory (Treg) cells. Hou et al. examined Blood samples from 57 never-smokers, 32 smokers with normal lung function and 66 patients with COPD, as well as bronchoalveolar lavage samples were taken from 12 never-smokers, 12 smokers and 18 patients with COPD. They found In peripheral blood, increased proportions of rTregs, aTregs and Fr III cells in smokers compared with never-smokers, whereas patients with COPD showed decreased rTregs and aTregs, and significantly increased Fr III cells compared with smokers. The changes in Treg subpopulations, with an overall decrease in the (aTreg+rTreg):(Fr III) ratio, indicated that immune homeostasis favoured inflammation and correlated with enhanced CD8 T-cell activation (r=-0.399, p<0.001) and forced expiratory volume in 1 s (FEV₁) % predicted value (r=0.435, p<0.001).The BAL (aTreg+rTreg):(Fr III) ratios displayed more robust correlations with FEV₁% predicted value (r=0.741, p<0.01) and activation of effector T cells(r=-0.763, p<0.001) [47]. Abnormalities of reduced Treg in COPD have also been described in animal models [48, 49].

In some embodiments of the invention, a patient presenting at a physician’s office or emergency department (ED) with COPD is administered mesenchymalstem cell or exosomes thereof. In further aspects, the patient is administered additional follow-on doses. Follow-on doses can be administered at various time intervals depending on the patient’s age, weight, ability to comply with physician instructions, clinical assessment, eosinophil count (blood or sputum eosinophils or eosinophilic cationic protein (ECP) measurement), or and other factors, including the judgment of the attending physician. The intervals between doses can be every 4 weeks, every 5 weeks, every 6 weeks, every 8 weeks, every 10 weeks, every 12 weeks, or longer intervals. In certain aspects, the intervals between doses can be every 4 weeks or every 8 weeks. In certain aspects, the intervals between doses can be every 4 weeks and every 8 weeks. In certain aspects, mesenchymal stem cell or exosomes thereof is administered with three four-week dosing intervals (i.e., on Day 0, Week 4, and Week 8) and then with eight-week dosing intervals (i.e., on Week 16, Week 24, Week 32, etc.).

In certain aspects, the single dose or first dose is administered to the COPD patient shortly after the patient presents with an acute exacerbation, e.g., a mild, moderate or severe exacerbation. For example, the single or first dose of mesenchymalstem cell or exosomes thereof can be administered during the presenting clinic or hospital visit, or in the case of very severe exacerbations, within 1, 2, 3, 4, 5, 6, 7, or more days, e.g., 7 days of the acute exacerbation, allowing the patient’s symptoms to stabilize prior to administration of mesenchymalstem cell or exosomes thereof.

In some embodiments, at least two doses of mesenchymalstem cell or exosomes thereof are administered to the patient. In some embodiments, at least three doses, at least four doses, at least five doses, at least six doses, or at least seven doses are administered to the patient. In some embodiments, mesenchymalstem cell or exosomes thereof is administered over the course of four weeks, over the course of eight weeks, over the course of twelve weeks, over the course of twenty-four weeks, over the course of forty-eight weeks, or over the course of a year or more.

The amount of mesenchymalstem cell or exosomes thereof to be administered to the patient can depend on various parameters such as the patient’s age, weight, clinical assessment, eosinophil count (blood or sputum eosinophils, eosinophilic cationic protein (ECP) measurement, or eosinophil derived neurotoxin (EDN) measurement), or and other factors, including the judgment of the attending physician. In certain aspects, the dosage or dosage interval is not dependent on the eosinophil level. In certain embodiments, the patient is administered one or more doses of mesenchymalstem cell or exosomes thereof wherein the dose is about 1-5 million cells per kilogram body weight.

In certain aspects, administration of mesenchymalstem cell or exosomes thereof according to the methods provided herein is through parenteral administration. For example, mesenchymalstem cell or exosomes thereof can be administered by intravenous infusion or by subcutaneous injection. In certain embodiments, mesenchymalstem cell or exosomes thereof can be administered by subcutaneous injection.

In certain aspects, mesenchymalstem cell or exosomes thereof is administered according to the methods provided herein in combination or in conjunction with additional therapies. Such therapies include, without limitation, corticosteroid therapy (including inhaled corticosteroids (ICS)), long-acting .beta. agonists (LABA, including long-acting (32 agonists), tiotropium, or other standard therapies. In certain aspects, benralizumab or an antigen-binding fragment there of is administered according to the methods provided herein in combination or in conjunction with ICS and LABA, with LABA and LAMA, or with ICS, LABA, and LAMA.

In certain instances, administration of mesenchymalstem cell or exosomes thereof decreases COPD exacerbations including, for example, as measured by an exacerbation rate, an annual exacerbation rate, time to first exacerbation, and/or an annual rate of COPD exacerbations that are associated with an emergency room visit or hospitalization.

The methods provided herein can reduce exacerbation rates in COPD patients. In certain aspects, use of the methods provided herein, i.e., administration of mesenchymalstem cell or exosomes thereof reduces the number of exacerbations experienced by the patient as compared to the number of exacerbations expected according to the patient’s history, as compared to the average number of exacerbations expected in a comparable population of patients, or as compared to a comparable population treated with placebo over the same time period. In certain aspects, administration of mesenchymalstem cell or exosomes thereof reduces the number of exacerbations in COPD patients with eosinophil counts of at least 200 eosinophils/.mu.L prior to the administration. In certain aspects, administration of mesenchymalstem cell or exosomes thereof reduces the number of exacerbations in COPD patients with eosinophil counts of at least 300 eosinophils/.mu.L prior to the administration. In certain aspects, administration of mesenchymalstem cell or exosomes thereof reduces the number of exacerbations in COPD patients with eosinophil counts of at least 400 eosinophils/.mu.L prior to the administration. In certain aspects, administration mesenchymalstem cell or exosomes thereof reduces the number of exacerbations in COPD patients with severe COPD as defined by the Global Initiative for Chronic Obstructive Lung Disease (GOLD), Global strategy for the diagnosis, management, and prevention of chronic obstructive pulmonary disease (updated 2009). In certain aspects, administration of mesenchymalstem cell or exosomes thereof reduces the number of exacerbations in COPD patients with very severe COPD as defined by the GOLD. In certain aspects, administration of mesenchymalstem cell or exosomes thereof reduces the number of exacerbations in COPD patients with severe or very severe COPD as defined by the GOLD. In certain aspects, administration of mesenchymalstem cell or exosomes thereof thereof reduces the number of exacerbations in COPD patients who are receiving corticosteroids (e.g., inhaled corticosteroids (ICS), long-acting .beta.-agonists (LABA) (e.g., long-acting .beta.2-agonists), and tiotropium.

In certain aspects, administration of mesenchymalstem cell or exosomes thereof reduces exacerbations by at least about 15%, by at least about 20%, by at least about 25%, by at least about 30%, at least about 35%, at least about 40%, at least about 45%, at least about 50%, or at least about 55%. In some embodiments, exacerbations are reduced about 34%, about 47%, or about 57%. The exacerbations can be reduced, for example, within a year from the first administration mesenchymalstem cell or exosomes thereof.

In certain aspects, use of the methods provided herein, i.e., administration of mesenchymalstem cell or exosomes thereof, reduces exacerbation rates within 4 weeks, within 8 weeks, within 12 weeks, within 16 weeks, within 20 weeks, within 24 weeks, within 28 weeks, within 32 weeks, within 36 weeks, within 40 weeks, within 44 weeks, within 48 weeks, or within 52 weeks. The methods provided herein can reduce exacerbation rates in COPD patients with severe or very severe COPD (as defined by GOLD), for example by at least 40% or by about 47%. The methods provided herein can reduce “annual exacerbation rates” in COPD patients

The methods provided herein can reduce the time to a first COPD exacerbation after a first administration of mesenchymalstem cell or exosomes thereof as compared to after a first administration of placebo. In some instances, administration of mesenchymalstem cell or exosomes thereof decreases the likelihood of a COPD exacerbation (e.g., within 52 weeks of a first administration of mesenchymalstem cell or exosomes thereof) as compared to the likelihood of a COPD exacerbation after treatment with placebo. In some instances, administration of mesenchymalstem cell or exosomes thereof decreases the annual rate of COPD exacerbations that are associated with an emergency room or hospitalization as compared to administration of placebo.

In certain instances, administration of mesenchymalstem cell or exosomes thereof improves the pulmonary function in a COPD patient, for example, as measured by forced expiratory volume in one second (FEV.sub.1) or forced vital capacity.

The methods provided herein can increase forced expiratory volume in one second (FEV.sub.1) in COPD patients. An increase can be measured based on the expected FEV.sub.1 based on a large patient population, on the FEV.sub.1 measured in a control population, or on the individual patient’s FEV.sub.1 prior to administration. In certain aspects, use of the methods provided herein, i.e., administration of mesenchymalstem cell or exosomes thereof, can increase the FEV.sub.1, as compared to the patient’s baseline FEV.sub.1. In some embodiments, the increased FEV.sub.1 is pre-bronchodilator FEV.sub.1. In some embodiments, the increased FEV.sub.1 is post-bronchodilator FEV.sub.1. In some embodiments, the increased FEV.sub.1 is pre-bronchodilator FEV.sub.1 and post-bronchodilator FEV.sub. 1. The FEV.sub.1 (e.g., the pre-bronchodilator and/or post-bronchodilator FEV.sub.1) can be increased, for example, within a year from the first administration of mesenchymalstem cell or exosomes thereof. For the use in the invention, a “bronchodilator,” as used herein, refers to any drug that widens or dilates the bronchi and bronchioles or air passages of the lungs, decreases resistance in the respiratory airway, and/or eases breathing by relaxing bronchial smooth muscle. For example, bronchodilators include short- and long-acting .beta.2-agonists such as albuterol/salbutamol and other drugs commonly used to treat asthma.

In certain embodiments of the invention, the methods provided herein can increase FEV.sub.1 by at least 5% or by at least 10%. In certain aspects, the methods provided herein can increase FEV.sub.1 by about 12%. In certain aspects, the methods provided herein can increase pre-bronchodilator FEV.sub.1 by at least 5% or by at least 10%. In certain aspects, the methods provided herein can increase pre-bronchodilator FEV.sub.1 by about 12%.

In certain aspects, the methods provided herein can increase FEV.sub.1 by at least 5%. In certain aspects, the methods provided herein can increase FEV.sub.1 by about 7%. In certain aspects, the methods provided herein can increase post-bronchodilator FEV.sub.1 by at least 5%. In certain aspects, the methods provided herein can increase post-bronchodilator FEV.sub.1 by about 7%.

In certain aspects, the methods provided herein can increase pre-bronchodilator and post-bronchodilator FEV.sub.1 by at least 5%. In certain aspects, the methods provided herein can increase can increase pre-bronchodilator by at least 10% and post-bronchodilator FEV.sub.1 by at least 5%. In certain aspects, the methods provided herein can increase pre-bronchodilator FEV.sub.1 by about 12% and post-bronchodilator FEV.sub.1 by about 7%. As provided herein, administration of mesenchymalstem cell or exosomes thereof can also increase the percent predicted FEV.sub.1 in COPD patients e.g., pre-bronchodilator and/or post-bronchodilator. By way of example, the percent predicted FEV.sub.1 can increase by about 3.0, about 3.5, about 4.0, or about 4.5. The methods provided herein can increase FEV.sub.1 in COPD patients with blood eosinophil counts of at least 200 eosinophils/.mu.L, or in patients receiving corticosteroids (e.g., inhaled corticosteroids (ICS), long-acting .beta.-agonists (LABA) (e.g., long-acting .beta.2-agonists), and tiotropium. In certain aspects, the methods provided herein can increase FEV.sub.1 in such patients by at least 10% or by at least 15%. In certain aspects, the methods provided herein can increase pre-bronchodilator FEV.sub.1 in such patients by at least 10% or by at least 15%. In certain aspects, the methods provided herein can increase post-bronchodilator FEV.sub.1 in such patients by about 10%. In certain aspects, the methods provided herein can increase pre-bronchodilator FEV.sub.1 and post-bronchodilator FEV.sub.1 in such patients by at least 10%. In certain aspects, the methods provided herein can increase pre-bronchodilator FEV.sub.1 in such patients by at least 15% and post-bronchodilator FEV.sub.1 in such patients by at least 10%. The methods provided herein can increase FEV.sub.1 in COPD patients with blood eosinophil counts of at least 300 eosinophils/.mu.L or in COPD patients with severe or very severe COPD as defined by the Global Initiative for Chronic Obstructive Lung Disease (GOLD). In certain aspects, the methods provided herein can increase FEV.sub.1 in such patients by at least 15% or by at least 20%. In certain aspects, the methods provided herein can increase pre-bronchodilator FEV.sub.1 in such patients by at least 15% or by at least 20%. In certain aspects, the methods provided herein can increase post-bronchodilator FEV.sub.1 in such patients by about 15%. In certain aspects, the methods provided herein can increase pre-bronchodilator FEV.sub.1 and post-bronchodilator FEV.sub.1 in such patients by at least 15%. In certain aspects, the methods provided herein can increase pre-bronchodilator FEV.sub.1 in such patients by at least 20% and post-bronchodilator FEV.sub.1 in such patients by at least 15%.

In certain aspects, use of the methods provided herein, i.e., administration of mesenchymalstem cell or exosomes thereof, increases the FEV.sub.1 within 4 weeks, within 8 weeks, within 12 weeks, within 16 weeks, within 20 weeks, within 24 weeks, within 28 weeks, within 32 weeks, within 36 weeks, within 40 weeks, within 44 weeks, within 48 weeks, within 52 weeks, or within 56 weeks or more. In certain aspects, administration of mesenchymalstem cell or exosomes thereof improves FEV.sub.1 within 52 weeks of a first administration of the mesenchymalstem cell or exosomes thereof. Use of the methods provided herein can increase FEV.sub.1 by at least 0.05 L, at least 0.1 L, at least 0.13 L, at least 0.15 L, at least 0.20 L, at least 0.21 L, at least 0.22 L, at least 0.23 L, at least 0.24 L, or at least 0.25 L, at least 0.30 L, at least 0.35 L, at least 0.40 L, at least 0.45 L, or at least 0.50 L over the 56-week period.

The methods provided herein can increase forced vital capacity (FVC) in COPD patients. An increase can be measured based on the expected FVC based on a large patient population, on the FVC measured in a control population, or on the individual patient’s FVC prior to administration. In certain aspects, use of the methods provided herein, i.e., administration of mesenchymalstem cell or exosomes thereof, can increase the FVC, as compared to the patient’s baseline FVC. In some embodiments, the increased FVC is pre-bronchodilator FVC. In some embodiments, the increased FVC is post-bronchodilator FVC. In some embodiments, the increased FVC is pre-bronchodilator FVC and post-bronchodilator FVC. The FVC (e.g., the pre-bronchodilator and/or post-bronchodilator FVC) can be increased, for example, within a year from the first administration of mesenchymalstem cell or exosomes thereof.

In certain aspects, the methods provided herein can increase FVC by at least 3%. In certain aspects, the methods provided herein can increase pre-bronchodilator FVC by at least 2%, at least 3%, at least 5% or at least 10%. In certain aspects, the methods provided herein can increase post-bronchodilator FVC by at least 2%, at least 3%, at least 5% or at least 10%. In certain aspects, the methods provided herein can increase pre-bronchodilator and post-bronchodilator FVC by at least 2%, at least 3%, at least 5% or at least 10%. In certain aspects, use of the methods provided herein, i.e., administration of mesenchymalstem cell or exosomes thereof, increases FVC within 4 weeks, within 8 weeks, within 12 weeks, within 16 weeks, within 20 weeks, within 24 weeks, within 28 weeks, within 32 weeks, within 36 weeks, within 40 weeks, within 44 weeks, within 48 weeks, within 52 weeks, or within 56 weeks or more.

In certain instances, administration of mesenchymalstem cell or exosomes thereof improves respiratory symptoms in a COPD patient, for example, as measured by the Baseline/Transitional Dyspnea Index (BDI/TDI) and/or the Exacerbations of Chronic Pulmonary Disease Tool-Respiratory Symptoms (E-RS).

Provided herein are also methods for improving respiratory symptoms as measured by the Baseline/Transitional Dyspnea Index (TDI). For example, administration of mesenchymalstem cell or exosomes thereof can improve (increase) a COPD patient’s BDI score by at least 1, at least 2, or at least 3 and/or result in a positive TDI score. The BDI/TDI score can be improved, for example, within a year from the first administration of mesenchymalstem cell or exosomes thereof.

In certain aspects, use of the methods provided herein, i.e., administration of mesenchymalstem cell or exosomes thereof, improves a BDI/TDI score within 4 weeks, within 8 weeks, within 12 weeks, within 16 weeks, within 20 weeks, within 24 weeks, within 28 weeks, within 32 weeks, within 36 weeks, within 40 weeks, within 44 weeks, within 48 weeks, within 52 weeks, or within 56 weeks or more.

Provided herein are also methods for improving respiratory symptoms as measured by the Exacerbations of Chronic Pulmonary Disease Tool-Respiratory Symptoms (E-RS). For example, administration of mesenchymalstem cell or exosomes thereof can improve (decrease) a COPD patient’s E-RS score by least 3, at least 4, at least 6, at least 7, at least 8, at least 9, or at least 10. The E-RS score can be improved, for example, within a year from the first administration of mesenchymalstem cell or exosomes thereof.

In certain aspects, use of the methods provided herein, i.e., administration of mesenchymalstem cell or exosomes thereof, improves a E-RS score within 4 weeks, within 8 weeks, within 12 weeks, within 16 weeks, within 20 weeks, within 24 weeks, within 28 weeks, within 32 weeks, within 36 weeks, within 40 weeks, within 44 weeks, within 48 weeks, within 52 weeks, or within 56 weeks or more.

In certain instances, administration of mesenchymalstem cell or exosomes thereof improves the health status and/or health-related quality of life in a COPD patient, for example, as measured by the Saint George’s Respiratory Questionnaire (SGRQ), the COPD-Specific Saint George’s Respiratory Questionnaire (SGRQ-C), and/or the COPD assessment tool (CAT).

Provided herein are methods for improving COPD symptoms, e.g., as assessed using a COPD questionnaire such as the Saint George’s Respiratory Questionnaire (SGRQ). For example, administration of mesenchymalstem cell or exosomes thereof can improve a patient’s SGRQ score by at least 3, at least 4, at least 6, at least 7, at least 8, at least 9, or at least 10. The SGRQ score can be improved, for example, within a year from the first administration of mesenchymalstem cell or exosomes thereof.

In certain aspects, use of the methods provided herein, i.e., administration of mesenchymalstem cell or exosomes thereof, improves a SGRQ score within 4 weeks, within 8 weeks, within 12 weeks, within 16 weeks, within 20 weeks, within 24 weeks, within 28 weeks, within 32 weeks, within 36 weeks, within 40 weeks, within 44 weeks, within 48 weeks, within 52 weeks, or within 56 weeks or more. In certain aspects, administration of mesenchymalstem cell or exosomes thereof improves an SGRQ score within 52 weeks of a first administration of the mesenchymalstem cell or exosomes thereof.

Provided herein are also methods for improving COPD symptoms, e.g., as assessed using a COPD questionnaire such as the COPD-Specific Saint George’s Respiratory Questionnaire (SGRQ-C). For example, administration of mesenchymalstem cell or exosomes thereof can improve a COPD patient’s SGRQ-C (symptom) score by at least 3, at least 4, at least 6, at least 7, at least 8, at least 9, or at least 10. The SGRQ-C (symptom) score can be improved, for example, within a year from the first administration of mesenchymalstem cell or exosomes thereof.

In certain aspects, use of the methods provided herein, i.e., administration of mesenchymalstem cell or exosomes thereof, improves a SGRQ-C (symptom) score within 4 weeks, within 8 weeks, within 12 weeks, within 16 weeks, within 20 weeks, within 24 weeks, within 28 weeks, within 32 weeks, within 36 weeks, within 40 weeks, within 44 weeks, within 48 weeks, within 52 weeks, or within 56 weeks or more.

Provided herein are also methods for improving COPD symptoms, e.g., as assessed using the COPD assessment tool (CAT). For example, administration of mesenchymalstem cell or exosomes thereof can improve (decrease) a COPD patient’s CAT score by least 3, at least 4, at least 6, at least 7, at least 8, at least 9, or at least 10. The CAT score can be improved (decreased), for example, within a year from the first administration of mesenchymalstem cell or exosomes thereof.

In certain aspects, use of the methods provided herein, i.e., administration of mesenchymalstem cell or exosomes thereof, improves (decreases) a CAT score within 4 weeks, within 8 weeks, within 12 weeks, within 16 weeks, within 20 weeks, within 24 weeks, within 28 weeks, within 32 weeks, within 36 weeks, within 40 weeks, within 44 weeks, within 48 weeks, within 52 weeks, or within 56 weeks or more.

In certain aspects, use of the methods provided herein, i.e., administration of mesenchymalstem cell or exosomes thereof, reduces nocturnal awakenings.

In certain aspects, use of the methods provided herein, i.e., administration of mesenchymalstem cell or exosomes thereof, reduces the use of rescue medication.

In certain aspects, use of the methods provided herein, i.e., administration of mesenchymalstem cell or exosomes thereof, reduces the severity, frequency, and/or duration of EXACT-PRO defined events.

In certain aspects, use of the methods provided herein, i.e., administration of mesenchymalstem cell or exosomes thereof, reduces COPD-specific resource utilization. For example, administration of mesenchymalstem cell or exosomes thereof can reduce unscheduled physician visits, unscheduled phone calls to physicians, and/or use of other COPD medications.

In certain aspects, use of the methods provided herein, i.e., administration of mesenchymalstem cell or exosomes thereof to a COPD patient, increases forced expiratory volume in one second (FEV.sub.1), increases forced vital capacity (FVC), reduces COPD exacerbation rate, and/or improves a COPD questionnaire score (e.g., the COPD control questionnaire).

In certain aspects, use of the methods provided herein, i.e., administration of mesenchymalstem cell or exosomes thereof to a COPD patient, decreases annual COPD exacerbation rate, improves SGRQ scores, and increases FEV.sub.1 (e.g., in COPD patients with a baseline blood eosinophil count .gtoreq.300/.mu.L).

In certain aspects, the COPD patient was prescribed or has been using corticosteroids (e.g., inhaled corticosteroids (ICS)), long-acting .beta.-agonists (LABA, e.g., long-acting .beta.2-agonists), and tiotropium prior to the administration of mesenchymalstem cell or exosomes thereof. In certain aspects, the COPD patient is treated with corticosteroids (e.g., ICS), LABA (e.g., long-acting .beta.2-agonists), tiotropium, and mesenchymalstem cell or exosomes thereof. In certain aspects, the COPD patient is treated with ICS and LABA. In certain aspects, the COPD patient is treated with LABA and long-acting muscarinic antagonist (LAMA). In certain aspects, the COPD patient is treated with ICS and LABA or with LABA and LAMA. In certain aspects, the COPD patient is treated with ICS, LABA, and LAMA.

In certain aspects of the methods provided herein, the patient has a history of COPD exacerbations. In certain aspects, the history of exacerbations comprises at least one exacerbation in the year prior to the administration of mesenchymalstem cell or exosomes thereof. In certain aspects, the patient has a forced expiratory volume (FEV.sub.1) of less than 80% predicted value prior to the administration. In certain aspects, the patient has an FEV.sub. ⅟FVC of less than 0.70 prior to the administration.

In one embodiment of the invention, exosomes are purified from mesenchymal stem cells by obtaining a mesenchymal stem cell conditioned medium, concentrating the mesenchymal stem cell conditioned medium, subjecting the concentrated mesenchymal stem cell conditioned medium to size exclusion chromatography, selecting UV absorbent fractions at 220 nm, and concentrating fractions containing exosomes.

Exosomes, also referred to as “particles” may comprise vesicles or a flattened sphere limited by a lipid bilayer. The particles may comprise diameters of 40-100 nm. The particles may be formed by inward budding of the endosomal membrane. The particles may have a density of .about.1.13-1.19 g/ml and may float on sucrose gradients. The particles may be enriched in cholesterol and sphingomyelin, and lipid raft markers such as GM1, GM3, flotillin and the src protein kinase Lyn. The particles may comprise one or more proteins present in mesenchymalstem cell or mesenchymalstem cell conditioned medium such as a protein characteristic or specific to the MESENCHYMALSTEM CELL or MESENCHYMALSTEM CELL-CM. They may comprise RNA, for example miRNA. Said particles may possess one or more genes or gene products found in Mesenchymalstem cell or medium which is conditioned by culture of Mesenchymalstem cell. The particle may comprise molecules secreted by the MESENCHYMALSTEM CELL. Such a particle, and combinations of any of the molecules comprised therein, including in particular proteins or polypeptides, may be used to supplement the activity of, or in place of, the Mesenchymalstem cell or medium conditioned by the Mesenchymalstem cell for the purpose of for example treating or preventing a disease. Said particle may comprise a cytosolic protein found in cytoskeleton e.g. tubulin, actin and actin-binding proteins, intracellular membrane fusions and transport e.g. annexins and rab proteins, signal transduction proteins e.g. protein kinases, 14-3-3 and heterotrimeric G proteins, metabolic enzymes e.g. peroxidases, pyruvate and lipid kinases, and enolase-1 and the family of tetraspanins e.g. CD9, CD63, CD81 and CD82. In particular, the particle may comprise one or more tetraspanins. The particles may comprise mRNA and/or microRNA. In one embodiment, mesenchymalstem cell exosomes, or particles may be produced by culturing mesenchymal stem cells in a medium to condition it. The mesenchymal stem cells may comprise human umbilical tissue derived cells which possess markers selected from a group comprising of CD90, CD73 and CD105. The medium may comprise DMEM. The DMEM may be such that it does not comprise phenol red. The medium may be supplemented with insulin, transferrin, or selenoprotein (ITS), or any combination thereof. It may comprise FGF2. It may comprise PDGF AB. The concentration of FGF2 may be about 5 ng/ml FGF2. The concentration of PDGF AB may be about 5 ng/ml. The medium may comprise glutamine-penicillin-streptomycin or b-mercaptoethanol, or any combination thereof. The cells may be cultured for about 1, 2, 3, 4, 5, 6, 7, 8, 9, 10 days or more, for example 3 days. The conditioned medium may be obtained by separating the cells from the medium. The conditioned medium may be centrifuged, for example at 500 g. it may be concentrated by filtration through a membrane. The membrane may comprise a >1000 kDa membrame. The conditioned medium may be concentrated about 50 times or more. The conditioned medium may be subject to liquid chromatography such as HPLC. The conditioned medium may be separated by size exclusion. Any size exclusion matrix such as Sepharose may be used. As an example, a TSK Guard column SWXL, 6.times.40 mm or a TSK gel G4000 SWXL, 7.8.times.300 mm may be employed. The eluent buffer may comprise any physiological medium such as saline. It may comprise 20 mM phosphate buffer with 150 mM of NaCl at pH 7.2. The chromatography system may be equilibrated at a flow rate of 0.5 ml/min. The elution mode may be isocratic. UV absorbance at 220 nm may be used to track the progress of elution. Fractions may be examined for dynamic light scattering (DLS) using a quasi-elastic light scattering (QELS) detector. Fractions which are found to exhibit dynamic light scattering may be retained. For example, a fraction which is produced by the general method as described above, and which elutes with a retention time of 11-13 minutes, such as 12 minutes, is found to exhibit dynamic light scattering. The r.sub.h of particles in this peak is about 45-55 nm. Such fractions comprise mesenchymalstem cell particles such as exosomes.

### EXAMPLES

BALB/c mice were administered with 1 million of either bone marrow or adipose MSC or with JadiCells Intravenously at the same time as intraperitoneal injection of elastase at the indicated concentrations. After a period of 3 days animals were sacrificed and concentration of neutrophils per viewing field was assessed. Animals receiving JadiCells had higher levels of MerTK and lower levels of TLR4.

### REFERENCES

ADDIN EN.REFLIST 1. Mapel, D.W., et al., Identifying and characterizing COPD patients in US managed care. A retrospective, cross-sectional analysis of administrative claims data. BMC Health Serv Res, 2011. 11: p. 43.

2. Ejiofor, S. and A.M. Turner, Pharmacotherapies for COPD. Clin Med Insights Circ Respir Pulm Med, 2013. 7: p. 17-34.

3. Miravitlles, M., et al., Costs of chronic obstructive pulmonary disease in relation to compliance with guidelines: a study in the primary care setting. Ther Adv Respir Dis, 2013. 7(3): p. 139-50.

4. Holloway, R.A. and L.E. Donnelly, Immunopathogenesis of chronic obstructive pulmonary disease. Curr Opin Pulm Med, 2013. 19(2): p. 95-102.

5. Motz, G.T., et al., Persistence of lung CD8 T cell oligoclonal expansions upon smoking cessation in a mouse model of cigarette smoke-induced emphysema. J Immunol, 2008. 181(11): p. 8036-43.

6. Maeno, T., et al., CD8+ T Cells are required for inflammation and destruction in cigarette smoke-induced emphysema in mice. J Immunol, 2007. 178(12): p. 8090-6.

7. Woodruff, P.G., et al., A distinctive alveolar macrophage activation state induced by cigarette smoking. Am J Respir Crit Care Med, 2005. 172(11): p. 1383-92.

8. Stefanska, A.M. and P.T. Walsh, Chronic obstructive pulmonary disease: evidence for an autoimmune component. Cell Mol Immunol, 2009. 6(2): p. 81-6.

9. Gonzalez-Rey, E., et al., Human adipose-derived mesenchymal stem cells reduce inflammatory and T cell responses and induce regulatory T cells in vitro in rheumatoid arthritis. Ann Rheum Dis. 69(1): p. 241-8.

10. Lepelletier, Y., et al., Galectin-1 and Semaphorin-3A are two soluble factors conferring T cell immunosuppression to bone marrow mesenchymal stem cell. Stem Cells Dev, 2009.

11. Tsyb, A.F., et al., In vitro inhibitory effect of mesenchymal stem cells on zymosan-induced production of reactive oxygen species. Bull Exp Biol Med, 2008. 146(1): p. 158-64.

12. Sun, L., et al., Mesenchymal stem cell transplantation reverses multiorgan dysfunction in systemic lupus erythematosus mice and humans. Stem Cells, 2009. 27(6): p. 1421-32.

13. Aslam, M., et al., Bone marrow stromal cells attenuate lung injury in a murine model of neonatal chronic lung disease. Am J Respir Crit Care Med, 2009. 180(11): p. 1122-30.

14. van Haaften, T., et al., Airway delivery of mesenchymal stem cells prevents arrested alveolar growth in neonatal lung injury in rats. Am J Respir Crit Care Med, 2009. 180(11): p. 1131-42.

15. Seimetz, M., et al., Inducible NOS inhibition reverses tobacco-smoke-induced emphysema and pulmonary hypertension in mice. Cell, 2011. 147(2): p. 293-305.

16. Iho, S., et al., Nicotine induces human neutrophils to produce IL-8 through the generation of peroxynitrite and subsequent activation of NF-kappaB. J Leukoc Biol, 2003. 74(5): p. 942-51.

17. Tanni, S.E., et al., Smoking status and tumor necrosis factor-alpha mediated systemic inflammation in COPD patients. J Inflamm (Lond), 2010. 7: p. 29.

18. Weathington, N.M., et al., A novel peptide CXCR ligand derived from extracellular matrix degradation during airway inflammation. Nat Med, 2006. 12(3): p. 317-23.

19. Xu, X., et al., A self-propagating matrix metalloprotease-9 (MMP-9) dependent cycle of chronic neutrophilic inflammation. PLoS One, 2011. 6(1): p. e15781.

20. Shoshani, T., et al., Identification of a novel hypoxia-inducible factor 1-responsive gene, RTP801, involved in apoptosis. Mol Cell Biol, 2002. 22(7): p. 2283-93.

21. Yoshida, T., et al., Rtp801, a suppressor of mTOR signaling, is an essential mediator of cigarette smoke-induced pulmonary injury and emphysema. Nat Med, 2010. 16(7): p. 767-73.

22. Kamocki, K., et al., RTP801 is required for ceramide-induced cell-specific death in the murine lung. Am J Respir Cell Mol Biol, 2013. 48(1): p. 87-93.

23. Buckley, S., et al., The milieu of damaged alveolar epithelial type 2 cells stimulates alveolar wound repair by endogenous and exogenous progenitors. Am J Respir Cell Mol Biol, 2011. 45(6): p. 1212-21.

24. Medler, T.R., et al., Apoptotic sphingolipid signaling by ceramides in lung endothelial cells. Am J Respir Cell Mol Biol, 2008. 38(6): p. 639-46.

25. Thon, L., et al., Ceramide mediates caspase-independent programmed cell death. FASEB J, 2005. 19(14): p. 1945-56.

26. Chung, K.F., Cytokines as targets in chronic obstructive pulmonary disease. Curr Drug Targets, 2006. 7(6): p. 675-81.

27. Tuder, R.M. and T. Yoshida, Stress responses affecting homeostasis of the alveolar capillary unit. Proc Am Thorac Soc, 2011. 8(6): p. 485-91.

28. Sukkar, M.B., et al., Toll-like receptor 2, 3, and 4 expression and function in human airway smooth muscle. J Allergy Clin Immunol, 2006. 118(3): p. 641-8.

29. Pace, E., et al., TLR4 upregulation underpins airway neutrophilia in smokers with chronic obstructive pulmonary disease and acute respiratory failure. Hum Immunol, 2011. 72(1): p. 54-62.

30. Speletas, M., et al., Association of TLR4-T399I polymorphism with chronic obstructive pulmonary disease in smokers. Clin Dev Immunol, 2009. 2009: p. 260286.

31. Wang, J., et al., Differential activation of killer cells in the circulation and the lung: a study of current smoking status and chronic obstructive pulmonary disease (COPD). PLoS One, 2013. 8(3): p. e58556.

32. Tassi, I., J. Klesney-Tait, and M. Colonna, Dissecting natural killer cell activation pathways through analysis of genetic mutations in human and mouse. Immunol Rev, 2006. 214: p. 92-105.

33. Wortham, B.W., et al., NKG2D mediates NK cell hyperresponsiveness and influenza-induced pathologies in a mouse model of chronic obstructive pulmonary disease. J Immunol, 2012. 188(9): p. 4468-75.

34. Borchers, M.T., et al., The NKG2D-activating receptor mediates pulmonary clearance of Pseudomonas aeruginosa. Infect Immun, 2006. 74(5): p. 2578-86.

35. Borchers, M.T., et al., Sustained CTL activation by murine pulmonary epithelial cells promotes the development of COPD-like disease. J Clin Invest, 2009. 119(3): p. 636-49.

36. Lie, M.L., et al., Lung T lymphocyte trafficking and activation during ischemic acute kidney injury. J Immunol, 2012. 189(6): p. 2843-51.

37. Shen, X., et al., CD4 T cells promote tissue inflammation via CD40 signaling without de novo activation in a murine model of liver ischemia/reperfusion injury. Hepatology, 2009. 50(5): p. 1537-46.

38. Ichim, C.V., Revisiting immunosurveillance and immunostimulation: Implications for cancer immunotherapy. J Transl Med, 2005. 3(1): p. 8.

39. Fraschini, F., et al., Some aspects of “deep lung” cellular immunity in chronic bronchitis before and after therapy with tiopronin. Int J Clin Pharmacol Res, 1987. 7(2): p. 129-33.

40. Mattoli, S., et al., The role of CD8+ Th2 lymphocytes in the development of smoking-related lung damage. Biochem Biophys Res Commun, 1997. 239(1): p. 146-9.

41. Cosio, M.G. and A. Guerassimov, Chronic obstructive pulmonary disease. Inflammation of small airways and lung parenchyma. Am J Respir Crit Care Med, 1999. 160(5 Pt 2): p. S21-5.

42. Stankiewicz, W., et al., Cellular and cytokine immunoregulation in patients with chronic obstructive pulmonary disease and bronchial asthma. Mediators Inflamm, 2002. 11(5): p. 307-12.

43. Agostini, C., L. Trentin, and F. Adami, Chronic obstructive pulmonary disease (COPD): new insights on the events leading to pulmonary inflammation. Sarcoidosis Vasc Diffuse Lung Dis, 2003. 20(1): p. 3-7.

44. Glader, P., K. von Wachenfeldt, and C.G. Lofdahl, Systemic CD4+ T-cell activation is correlated with FEV1 in smokers. Respir Med, 2006. 100(6): p. 1088-93.

45. Di Stefano, A., et al., STAT4 activation in smokers and patients with chronic obstructive pulmonary disease. Eur Respir J, 2004. 24(1): p. 78-85.

46. Roos-Engstrand, E., et al., Cytotoxic T cells expressing the co-stimulatory receptor NKG2 D are increased in cigarette smoking and COPD. Respir Res, 2010. 11: p. 128.

47. Hou, J., et al., Imbalance between subpopulations of regulatory T cells in COPD. Thorax, 2013.

48. Wang, H., et al., Imbalance of Th17/Treg cells in mice with chronic cigarette smoke exposure. Int Immunopharmacol, 2012. 14(4): p. 504-12.

49. Eppert, B.L., et al., Functional characterization of T cell populations in a mouse model of chronic obstructive pulmonary disease. J Immunol, 2013. 190(3): p. 1331-40.

1. A method of treating a lung degenerative disease comprising administration of a therapeutic cell, wherein said therapeutic cell is generated by the steps of: a) obtaining umbilical cord mesenchymal stem cells; b) culturing said mesenchymal stem cells in a liquid media capable of allowing for proliferation of said mesenchymal stem cells; c) extracting from said single cell suspension cells expressing the markers CD31 and CD73; and d) priming said cells with an agent capable of augmenting production of lung regenerative properties of said cells.

2. The method of claim 1, wherein said lung regenerative properties are selected from a group comprising of: a) inhibiting inflammation; b) enhancing renewal of pulmonary progenitor cells; c) inhibiting pulmonary fibrosis; and d) preventing apoptosis of pulmonary cells.

3. The method of claim 2, wherein said mesenchymal stem cell are activated by exposure to a toll like receptor agonist.

4. The method of claim 1, wherein said umbilical cord mesenchymal stem cell is an isolated cell prepared by a process comprising: placing a subepithelial layer of a mammalian umbilical cord tissue in direct contact with a growth substrate; and culturing the subepithelial layer such that the isolated cell from the subepithelial layer is capable of self-renewal and culture expansion, wherein the isolated cell expresses at least three cell markers selected from the group consisting of CD29, CD73, CD90, CD166, SSEA4, CD9, CD44, CD146, or CD105, and wherein the isolated cell does not express NANOG and at least five cell markers selected from the group consisting of CD45, CD34, CD14, CD79, CD106, CD86, CD80, CD19, CD117, Stro-1, or HLA-DR.

5. The method of claim 4, wherein the isolated cell expresses CD29, CD73, CD90, CD166, SSEA4, CD9, CD44, CD146, and CD105.

6. The method of claim 4, wherein the isolated cell does not express CD45, CD34, CD14, CD79, CD106, CD86, CD80, CD19, CD117, Stro-1, and HLA-DR.

7. The method of claim 5, wherein the isolated cell is positive for SOX2.

8. The method of claim 4, wherein the isolated cell is positive for OCT4.

9. The method of claim 4, wherein the isolated cell is positive for SOX2 and OCT4.

10. The method of claim 4, wherein the wherein the isolated cell is capable of differentiation into a cell type selected from the group consisting of adipocytes, chondrocytes, osteocytes, cardiomyocytes, endothelial cells, and myocytes.

11. The method of claim 4, wherein the isolated cell produces exosomes expressing CD63, CD9, or CD63 and CD9.

12. The method of claim 4, wherein culturing comprises culturing in a culture media that is free of animal components.

13. The method of claim 4, wherein the isolated cell has been expanded into a cell culture.

14. The method of claim 13, wherein the expanded cell culture is a culture of differentiated cells that includes a cell type selected from the group consisting of adipocytes, chondrocytes, osteocytes, cardiomyocytes, endothelial cells, and myocytes.

15. The method of claim 1, wherein IL-2 is administered together with said mesenchymal stem cells.

16. The method of claim 15, wherein said IL-2 is administered at a concentration sufficient to induce generation of T regulatory cells.

17. The method of claim 16, wherein said T regulatory cells express FoxP3.

18. The method of claim 16, wherein said T regulatory cells express PD-1L.

19. The method of claim 16, wherein said T regulatory cells suppress death of type II pulmonary epithelial cells.

20. The method of claim 16, wherein said T regulatory cells suppress inflammation of type II pulmonary epithelial cells.
